(19) United States
(12) Patent Application Publication
(10) Pub. No.: US 2023/0170077 A1
(43) Pub. Date: Jun. 1, 2023

(54) MEDICAL INFORMATION DISPLAY APPARATUS AND METHOD

(71) Applicant: Canon Medical Systems Corporation, Otawara-shi (JP)

(72) Inventors: Keita MITSUMORI, Nasushiobara (JP); Takamasa NAGAO, Yaita (JP); Taisuke IWAMURA, Utsunomiya (JP); Junya SUZUKI, Utsunomiya (JP)

(73) Assignee: Canon Medical Systems Corporation, Otawara-shi (JP)

(21) Appl. No.: 18/058,341

(22) Filed: Nov. 23, 2022

(30) Foreign Application Priority Data
Nov. 29, 2021 (JP) 2021-193154

(51) Int. Cl. G16H 30/40

(52) U.S. Cl. CPC G16H 30/40

(57) ABSTRACT

A medical information processing apparatus includes processing circuitry. The processing circuitry obtains a plurality of execution results respectively corresponding to a plurality of analysis processes. The execution results are obtained by performing the plurality of analysis processes relating to at least one type of analysis application on a plurality of series of medical images. The processing circuitry displays a first display area for displaying the plurality of execution results which are summarized by a type of analysis application, and a second display area for displaying input images provided to the plurality of analysis processes. The input images are identifiably displayed for each type of analysis application.

### CROSS-REFERENCE TO RELATED APPLICATION(S)

This application is based upon and claims the benefit of priority from Japanese Patent Application No. 2021-193154, filed Nov. 29, 2021, the entire contents of which are incorporated herein by reference.

### FIELD

Embodiments described herein relate generally to a medical information display apparatus and method.

### BACKGROUND

A medical network system which automatically analyzes DICOM (digital imaging and communications in medicine) data, such as received medical images, etc., is known. In such a medical network system, an analysis application that matches conditions is run for each examination or series. This system runs the same analysis application only once for one examination.

### BRIEF DESCRIPTION OF THE DRAWINGS

FIG. 1 is a diagram showing a configuration example of a medical information processing system according to a first embodiment.

FIG. 2 is a diagram showing a configuration example of the medical information display apparatus according to the first embodiment.

FIG. 3 is a diagram showing the relationship between analysis processing achieved by applying an analysis application to each series and a result of the analysis processing.

FIG. 4 is a diagram showing a development of analysis processing and a result of the processing for each analysis application.

FIG. 5 is a diagram showing an example of a management screen (examination result display screen) caused to be displayed on a display terminal by the medical information display apparatus.

FIG. 6 is a diagram showing an example of a management screen (examination list display screen) caused to be displayed on a display terminal by the medical information display apparatus.

FIG. 7 is a diagram showing an example of a summarized status mark.

FIG. 8 is a diagram showing another example of a summarized status mark.

FIG. 9 is a diagram showing another example of a summarized status mark.

FIG. 10 is a diagram showing a configuration example of a medical information display apparatus according to a second embodiment.

FIG. 11 is a diagram showing a flow of a summarized status display process by the medical information display system according to the second embodiment.

FIG. 12 schematically shows the process of setting display content for the summarized status.

FIG. 13 is a diagram showing a presence/absence of administration of a tPA treatment under Japan guideline and Western guideline.

FIG. 14 is a diagram showing an example of displaying icons of analysis processing.

FIG. 15 is a diagram schematically showing changing of an examination result display screen between user A and user B according to the first modification.

FIG. 16 is a diagram showing development of an examination result display screen regarding user B shown in FIG. 15 as a result of pressing a publication button.

FIG. 17 is a diagram schematically showing an example of a display of a running time for each user according to a second modification.

### DETAILED DESCRIPTION

According to an embodiment, a medical information processing apparatus includes processing circuitry. Processing circuitry obtain a plurality of execution results respectively corresponding to a plurality of analysis processes, the execution results being obtained by performing the plurality of analysis processes relating to at least one type of analysis application on a plurality of series of medical images. Processing circuitry display a first display area for displaying the plurality of execution results which are summarized by a type of analysis application, and a second display area for displaying input images provided to the plurality of analysis processes on a display, the input images being identifiably displayed for each type of analysis application, the second display area differing from the first display area.

Embodiments of the medical information display apparatus and method will be described in detail below with reference to the accompanying drawings.

### First Embodiment

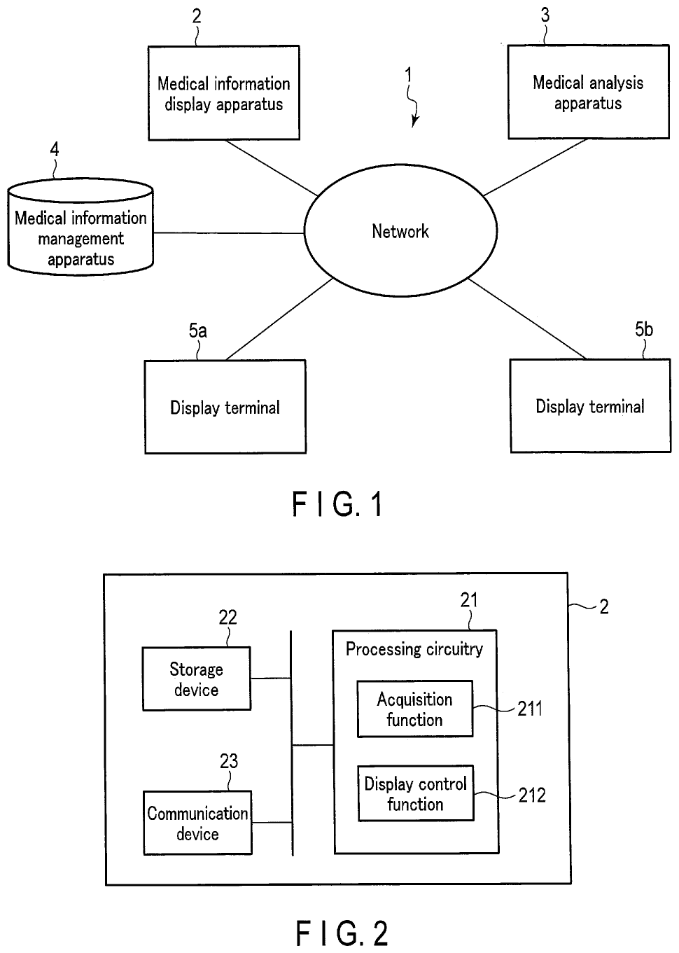

FIG. 1 is a diagram showing a configuration example of a medical information processing system 1 according to a first embodiment. As shown in FIG. 1, the medical information processing system 1 is a computer network system having a medical information processing apparatus 2, a medical analysis apparatus 3, a medical information management apparatus 4, a display terminal 5a, and a display terminal 5b. The medical information display apparatus 2, the medical analysis apparatus 3, the medical information management apparatus 4, the display terminal 5a, and the display terminal 5b are communicably connected to each other via a communication network, such as a LAN, etc. The medical information processing system 1 performs analysis processing relating to one or more types of analysis applications (hereinafter, “analysis app”) multiple times and causes an analysis result of the analysis processing performed multiple times to be displayed.

The medical information management apparatus 4 is a storage device storing DICOM data such as a medical image, etc. DICOM data is medical data, such as medical images and analysis results, etc., managed by the DICOM standard. DICOM data is managed in a hierarchical manner, being divided according to examinations and series. Medical images are image data generated by performing medical imaging on a subject by a medical image diagnosis apparatus. A type of medical image diagnosis apparatus (modality) is not particularly limited as long as a medical image of a subject can be obtained, and an X-ray computed tomography apparatus, an X-ray diagnosis apparatus, a magnetic resonance imaging apparatus, an ultrasound diagnosis apparatus, a nuclear medicine diagnostic apparatus, an endoscope examination apparatus, an optical coherence tomography imaging apparatus, etc., may be used.

The medical analysis apparatus 3 is a computer that performs analysis processing multiple times by running one or more types of analysis apps on a medical image. Data of results of each analysis process is transmitted to the medical information management apparatus 4 and/or the medical information display apparatus 2. The results herein include not only an analysis result of analysis processing but also a performance state, such as “successful”, “failed”, “being performed”, and “not yet executed”.

The display terminals 5a and 5b are a computer that functions as a client device of each of the medical information display apparatus 2, the medical analysis apparatus 3, and the medical information management apparatus 4. As an example, the display terminals 5a and 5b display a result on a display device. As the display device, for example, a liquid crystal display (LCD), a cathode ray tube (CRT) display, an organic electro luminescence display (OELD), a plasma display, or any other display can be used as appropriate. A projector may be used as the display device. As another example, the display terminals 5a and 5b transmit an instruction that is input by a user through an input device to, for example, the medical information display apparatus 2, the medical analysis apparatus 3, and the medical information management apparatus 4. As the input interface, for example, a mouse, a keyboard, a trackball, a switch, a button, a joystick, a touch pad, or a touch panel display can be used as appropriate. As the display terminals 5a and 5b, an installation-type computer, a laptop computer, a tablet device, a smartphone, etc., can be adopted. Three or more display terminals may be provided to the medical information processing system 1. In the description hereinafter, if there is no need to distinguish the display terminal 5a from the display terminal 5b, they are simply referred to as a “display terminal 5”.

FIG. 2 is a diagram showing a configuration example of the medical information display apparatus 2. As shown in FIG. 2, the medical information display apparatus 2 has processing circuitry 21, a storage device 22, and a communication device 23. The processing circuitry 21, the storage device 22, and the communication device 23 are connected to each other via a bus in such a manner that signals can be mutually input and output. The medical information display apparatus 2 is a computer that causes the display terminals 5a and 5b to display a result of performing each analysis processing.

The processing circuitry 21 includes a processor, such as a central processing unit (CPU). The processing circuitry 21 realizes an obtainment function 211 and a display control function 212 through execution of a medical information display program. Each of the functions 211 and 212 are not necessarily implemented by a single processing circuit. Processing circuitry may be composed by combining a plurality of independent processors, and the respective processors may execute programs, thereby realizing the functions 211 and 212. The functions 211 and 212 may be respective modularized programs constituting a medical information display program. These programs are stored in the storage device 22.

By the realization of the obtainment function 211, the processing circuitry 21 obtains various information items. For example, the processing circuitry 21 obtains from the medical information management apparatus 4 and/or the medical analysis apparatus 3, a plurality of results of a plurality of analysis processes, which can be obtained through performing a plurality of analysis processes relating to at least one type of analysis app on a plurality of series of medical images.

Through realization of the display control function 212, the processing circuitry 21 causes the display terminal 5 to display various information items. For example, the processing circuitry 21 causes the display terminal 5 to display a result of analysis processing. Specifically, the processing circuitry 21 causes the display terminal 5 to display a first display area for displaying a plurality of results which are summarized by types of analysis apps, and a second display area, which differs from the first display area, for displaying input images provided to the plurality of analysis processes in a manner such that the images can be identified for each analysis app type. The processing circuitry 21 causes the first display area to display identification information indicating the status of the analysis processing, “being performed”, “not yet performed”, “successful”, or “failed”, as a plurality of results. The processing circuitry 21 causes the first display area to display an analyzed image, which is a result of performing the analysis processing and/or input parameters of the analysis processing, as a plurality of results. The processing circuitry 21 causes the second display area to display a plurality of representative images respectively corresponding to a plurality of series, with an emphasis on a representative image of a series on which an analysis process selected from a plurality of analysis processes is performed.

The storage device 22 is a ROM (read only memory), a RAM (random access memory), an HDD (hard disk drive), an SSD (solid state drive), or an integrated circuit storage device, etc. storing various types of information. The storage device 22 may be not only the above-listed storage devices, but also a driver that writes and reads various types of information to and from, for example, a portable storage medium such as a compact disc (CD), a digital versatile disc (DVD), a flash memory, or a semiconductor memory. The storage device 22 may be provided in another computer connected via a network.

The communication device 23 is an interface for sending and receiving various types of information to and from other computers, such as the medical analysis apparatus 3, the medical information management apparatus 4, the display terminal 5, etc. included in the medical information processing system 1.

Hereinafter, an operation example of the medical information processing system 1 according to the first embodiment is described.

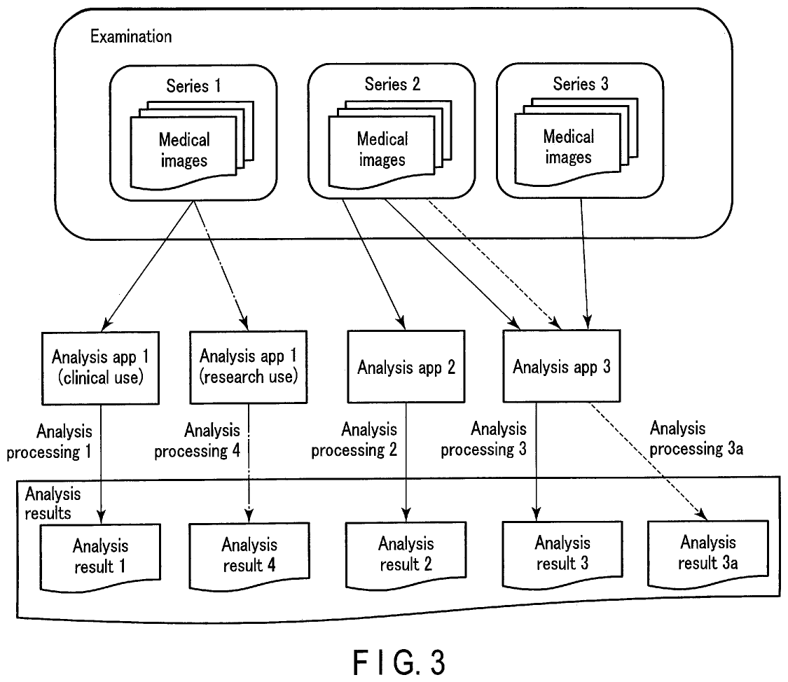

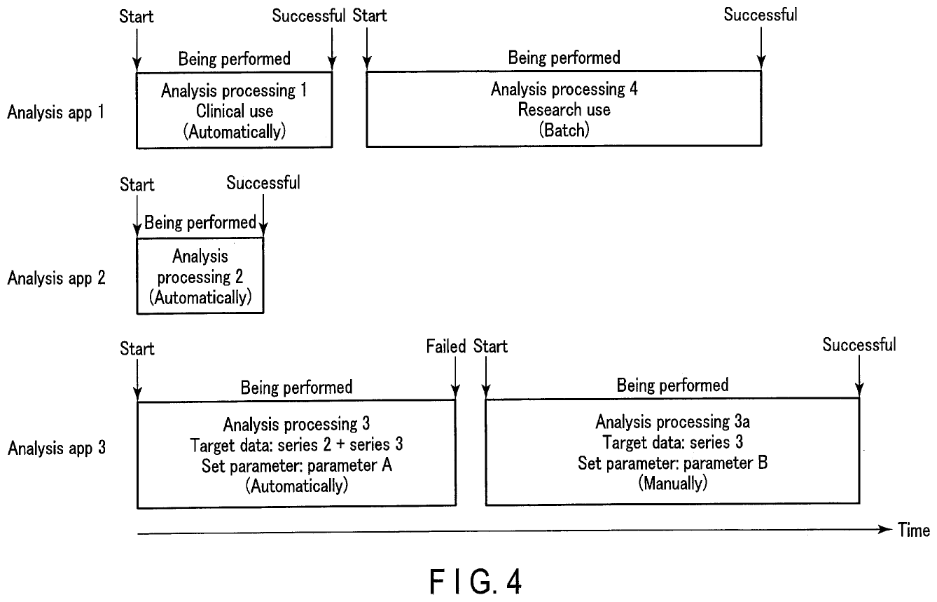

First, referring to FIGS. 3 and 4, the plurality of analysis processes performed by the medical information processing system 1 by using a plurality of analysis apps are described. FIG. 3 is a diagram showing the relationship between analysis processing achieved by applying an analysis application to each series and a result of the analysis processing. FIG. 4 is a diagram showing a development of analysis processing and a result of performing the processing for each analysis application. Suppose each examination includes three series (series 1, series 2, and series 3) in the example shown in FIGS. 3 and 4. Suppose each series includes data of a plurality of medical images.

As shown in FIG. 3, three series of medical images of a subject are subsequently collected by a medical image diagnosis apparatus. These three series of medical images may be collected by a medical image diagnosis apparatus of the same modality or medical image diagnosis apparatuses of different modalities. Each medical image in the three series is automatically and instantly transmitted from the medical image diagnosis apparatus to the medical information management apparatus 4. The data of each medical image of the three series may be transmitted to the medical information management apparatus 4 after being subjected to image checking to see if an image quality suitable for an examination order is attained or not. The medical information management apparatus 4 stores the three series of medical images that are received. In parallel with this, the medical information management apparatus 4 automatically and instantly transmits these three series of medical images to the medical analysis apparatus 3.

The medical analysis apparatus 3 automatically performs analysis processing on each medical image in the received three series by applying a preset analysis app. As shown in FIGS. 3 and 4, in the present embodiment, suppose there are three analysis apps (analysis app 1, analysis app 2, analysis app 3) used for different purposes. A specific content of each analysis app is not particularly limited, and it suffices that analysis processing is performed according to various uses. For example, there is an analysis app with which an analysis of bleeding is performed, an analysis app with which an analysis of ischemia is performed, and an analysis app with which an analysis of a lesion, such as a tumor, is performed.

As shown in FIGS. 3 and 4, as an example, the medical analysis apparatus 3 automatically performs analysis processing 1 by applying analysis app 1 to the medical images of series 1. A processing algorithm and input parameters of analysis processing 1 are for clinical use, and as analysis processing 1, an analysis with a high reliability is performed in compliance with determination criteria adopted by an organization that conducts the analysis processing. Assume analysis result 1 is output as a result of analysis processing 1. Assume analysis processing 1 is used for a clinical purpose. The medical analysis apparatus 3 automatically performs analysis processing 2 by applying the medical images of series 2 to analysis app 2. Assume analysis result 2 is output as a result of analysis processing 2. The medical analysis apparatus 3 automatically performs analysis processing 3 by applying analysis app 3 to the medical images of series 2 and the medical images of series 3. Assume analysis result 3 is output as a result of analysis processing 3.

During each analysis process, the medical analysis apparatus 3 transmits data of the result of the analysis processing to the medical information display apparatus 2. A result includes not only an analysis result that is output when analysis processing is successful but also a performance state of the analysis processing, such as “successful”, “failed”, “being performed”, or “not yet performed”, etc. A parameter value calculated by image analysis and an analyzed image in which the parameter value is assigned to a pixel and the like may be obtained as analysis results. The medical analysis apparatus 3 may actively transmit the result data to the medical information display apparatus 2, or passively transmit it to the medical information display apparatus 2 in response to a transmit request from the medical information display apparatus 2. A timing of transmitting result data is not particularly limited; it suffices that the result data is transmitted for each analysis app and/or each analysis process every predetermined length of time or every time content of the results changes.

Suppose analysis processing 1 is successful for analysis app 1, as shown in FIGS. 3 and 4. If analysis processing 1 is successful, the medical analysis apparatus 3 automatically performs analysis processing 4 by applying the medical images of series 1 to analysis app 1. The performance of analysis processing 4 may be set in advance so that the processing can be automatically started upon successful performance of analysis processing 1, or may be started in response to an instruction to start that is input by a user via the display terminal 5, etc. The processing algorithm and input parameters of analysis processing 1 are used for research, and analysis processing 4 is batch.

Herein assume analysis processing 2 is successful for analysis app 2, as shown in FIGS. 3 and 4. Assume no analysis processing is performed thereafter for analysis app 2.

Herein assume analysis processing 3 failed for analysis app 3, as shown in FIGS. 3 and 4. Various reasons for the failure may be assumed, for example, temporary memory shortage, erroneous input of processing target data, or malfunction of hardware, etc. If analysis processing 3 failed, the medical analysis apparatus 3 once again runs the analysis app 3 to perform analysis processing 3a. To avoid another failure, analysis processing 3a is performed under performing conditions differing from those for analysis processing 3, such as input parameters and processing target data, etc. For example, suppose the target data of analysis processing 3 is the medical images of series 2 and series 3, and the input parameter is a parameter A, and target data of the analysis processing 3a is medical images of series 3, and the input parameter is a parameter B. Target data and input parameters are by a user via the display terminal 5, and in response to an absence of an instruction to start, the medical analysis apparatus 3 performs analysis processing 3a based on the target data and the input parameters. Assume analysis processing 3a is successful.

The medical information display apparatus 2 causes the display terminal 5 to instantly display a management screen for showing a result of each analysis process based on the analysis processing results received from the medical analysis apparatus 3. Specifically, the processing circuitry 21 obtains, through realization of the obtainment function, a result of each analysis process transmitted from the medical analysis apparatus 3, and through the display control function 212, generates a management screen for displaying a list of the obtained results of the analysis processing, and causes the display terminal 5 to display the generated management screen.

As described above, the medical information processing system 1 according to the present embodiment can run the same analysis app multiple times for the same examination. As a circumstance where the same analysis app is run multiple times, cases where the processing fails and the same app is run again as described above, the same app is run with different input parameters, different analyses are required for clinical use and research use, and the like are assumed.

If the same analysis app is run multiple times for the same examination, there is a possibility that analysis results and results of performing the analysis processing will not be able to be appropriately managed on the management screen, compared to the case where the same analysis app is run only once for the same examination. For example, since only the results of running the analysis app are put in a chronological order, it is assumed that comparison of the input parameters and the target data between different analysis processes using the same analysis app may be difficult. To address such a situation, the medical information processing system 1 provides a management screen with good viewability for results of analysis processing when the same analysis app is run multiple times for the same examination.

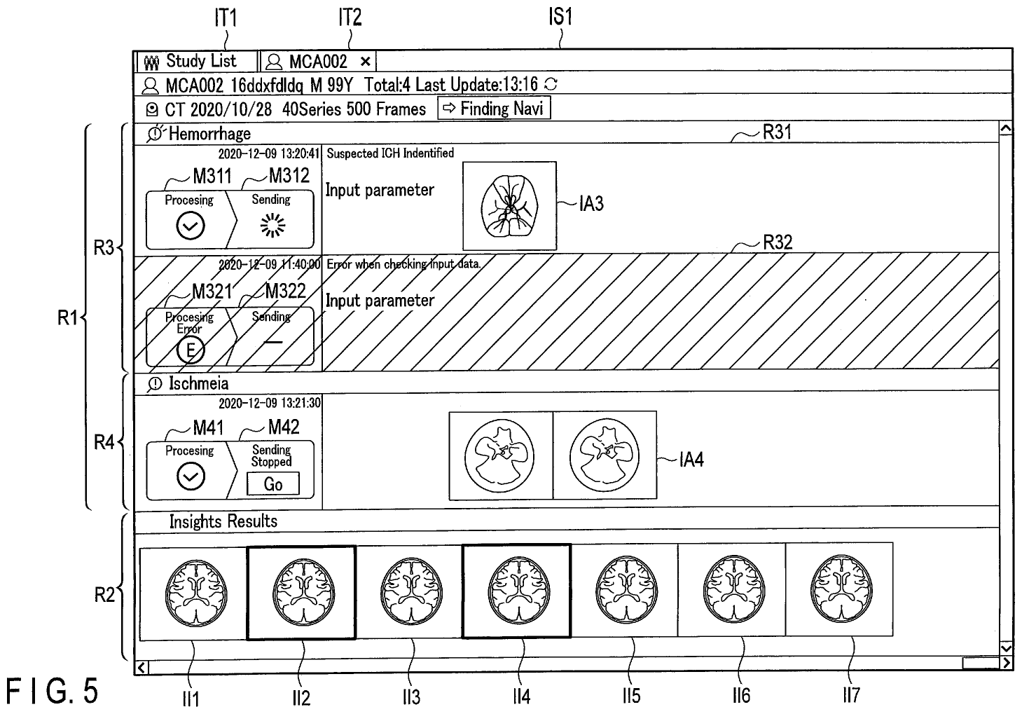

FIG. 5 is a diagram showing an example of a management screen (examination result display screen) IS1 caused to be displayed on a display terminal 5 by the medical information display apparatus 2. In the management screen IS1, an examination list tab Ill and an examination result display tab IT2 are provided. If the examination list tab IT1 is selected, a display screen for an examination list is displayed on the management screen IS1, and if one examination in the examination list is selected, the examination results corresponding to the selected examination are displayed on a sectioned management screen IS1 in the examination result display tab IT2 (hereinafter, an “examination result display screen IS1”).

As shown in FIG. 5, the processing circuitry 21 causes a result display area R1 and an input image display area R2 to be displayed side by side on one screen. The result display area R1 displays one or more analysis processes in a list for each analysis app. For example, in the result display area R1, a display area R3 for a result of running the analysis app “Hemorrhage” and a display area R4 for a result of running the analysis app “Ischemia” is arranged. In the display area R3, multiple analysis processes relating to the analysis app “Hemorrhage” are arranged by predetermined rules regarding processing order. For example, two display areas R31 and R32 are arranged in the display area R3 in reverse chronological order of processing. Assume a situation where the first analysis process failed and the second analysis process was therefore performed.

As shown in FIG. 5, results and processing conditions of the analysis process are displayed in the display areas R31 and R32. The performance state and the analyzed images, the input parameters of the analysis processing, etc. are displayed as the result. The performance state is expressed by identification information indicating whether the analysis processing is being performed, has not yet been performed, is successful, or has failed. As an example of identification information, a mark M321 indicating the performance state “failed” is displayed in the display area R32. Since the analysis processing failed, the analyzed image is not displayed in the display area R32. In the display area R31, a mark M311 indicating a running performance of “successful” is displayed, as an example of identification information. Since the analysis processing is successful, an analyzed image IA3 is displayed in the display area R31. As another example of identification information, the mark M322 indicating the sending state “not transmitted yet” may be displayed in the display area R32, and the mark M312 indicating the sending state “transmitting” may be displayed in the display area R31. The sending state indicates a sending state of an analyzed image from the medical analysis apparatus 3 to the medical information management apparatus 4 or the medical information display apparatus 2.

As shown in FIG. 5, since the analysis app “Ischemia” is run only once, the display area R4 relating to a single analysis process is displayed. In the display area R4, a mark M41 indicating that the performance state is successful is displayed, and a mark M42 indicating that the sending state is “already transmitted” is displayed. Since the analysis processing is successful, an analyzed image IA4 is displayed in the display area R4.

As shown in FIG. 5, a plurality of representative images respectively corresponding to multiple series included in the examination are displayed in the input image display area R2. In other words, the plurality of representative images means the images that are input to the plurality of analysis apps. As the representative images, a thumbnail of a single medical image selected based on a predetermined criterion from a plurality of medical images included in the series is used. The medical image selected based on a predetermined criterion may be either a medical image selected by a user or a medical image automatically selected according to a time of imaging or a purpose of a series.

As shown in FIG. 5, an analysis process can be discretionarily selected by a user via the display terminal 5. The display area for the selected analysis process is visually emphasized by display effects such as a box and color. In FIG. 5, the display area R32 is indicated by a box. If an analysis process is thus selected, a thumbnail image corresponding to an input image of the selected analysis process is emphasized in the input image display area R2. In FIG. 5, the thumbnail images 112 and 114 are emphasized as input images for the first running of the analysis app “Hemorrhage”. It is thereby possible to easily know input images in the selected analysis process.

The above-described management screen IS1 is merely an example, and it can be modified in various ways. In the foregoing example, the processing circuitry 21 causes the result display area R1 and the input image display area R2 to be displayed side by side on one screen. However, the processing circuitry 21 may cause the result display area R1 and the input image display area R2 to be displayed separately. The processing circuitry 21 may not necessarily cause both of the analysis images and the input parameters to be displayed, and may cause either of them to be displayed. In the foregoing example, a performance state is indicated by a mark; however, it may be indicated by a text, etc.

As described above, since the results of running the same analysis app are summarized and displayed on the management screen IS1, it is possible to easily compare a plurality of analysis processes performed by running the same analysis app. For example, a user can confirm at a glance that a first analysis process for the analysis app “Hemorrhage” failed but the second one is successful. Furthermore, since the input parameters, analyzed images, input images (representative images) are displayed for both the first and second analysis processes, it is possible to use these pieces of displayed content to consider reasons why the first analysis processing failed or the second analysis processing was successful. Even when the first and second analysis processing are both successful, it is possible to consider differences in the analysis results brought about by differences in the input parameters and the input images. This is also applicable to the case where both the first and second analysis processes failed.

Next, the management screen on which an examination list is displayed by the examination list tab IT1 (hereinafter, an “examination list display screen”) is described. The processing circuitry 21 causes, through realization of the display control function 212, the display terminal 5 to display a third display area (examination list display screen) on which a list of examination records respectively corresponding to multiple examinations is displayed. If one examination record is selected from a plurality of examination records, the processing circuitry 21 causes the first display area (examination display area) and the second display area (input image display area) relating to the selected examination record to be displayed. For each of the examination records, the processing circuitry 21 causes a mark on which a plurality of performance states relating to multiple examination records are summarized to be displayed.

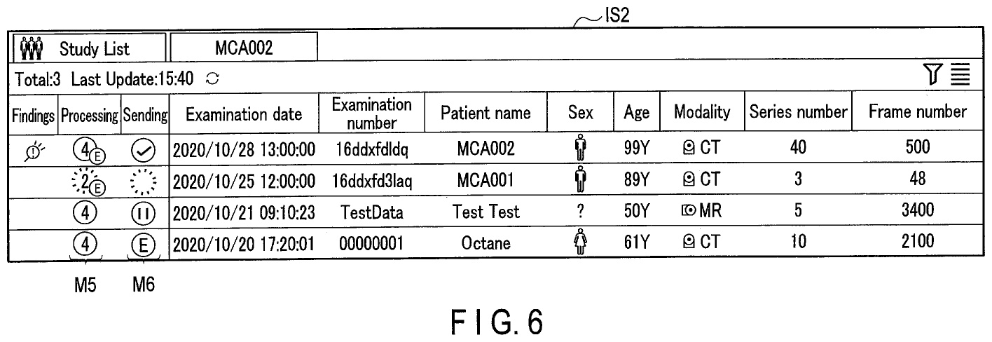

FIG. 6 is a diagram showing an example of a management screen (examination list display screen) IS2 caused to be displayed on the display terminal 5 by the medical information display apparatus 2. The examination list display screen IS2 is displayed on the display terminal 5 when the examination list tab IT1 is selected.

As shown in FIG. 6, the examination list display screen IS2 shows a list of examinations managed by the medical information management apparatus 4. Specifically, a plurality of examination records respectively corresponding to the plurality of examinations are displayed in a list. Each examination record is displayed with values of items, for example: “findings” indicating whether or not findings should be generated; “processing” indicating a summarized status of results; “sending” indicating a data sending state; “examination date” indicating a date of examination; “name of patient” indicating designation of a patient or a patient's ID; “sex” indicating sex of a patient; “age” indicating an age of a patient; “modality” indicating a type of modality with which an examination is performed; “series number” indicating the number of series included in an examination; “frame number” indicating the number of medical images collected by an examination, and so on. If a single examination record is selected from a plurality of examination records via a display terminal 5, etc., results of the examination (hereinafter a “display-targeted examination”) corresponding to the selected examination record are displayed on the examination result display screen shown in FIG. 5, etc.

The summarized status displayed in the item “processing” is a summarization of a plurality of performance states corresponding to the plurality of analysis processes belonging to the examination. The mark indicating a summarization status, namely, a mark M5 on which a plurality of performance states corresponding to a plurality of analysis processes belonging to the examination are summarized (hereinafter a “summarized status mark”), is displayed in the “processing” section. The summarized status mark M5 indicates the number of analysis processes belonging to the examination, and whether the analysis processing is being performed, has not yet been performed, is successful, or has failed.

As shown in FIG. 6, the mark indicating the sending state of an analysis result from the medical analysis apparatus 3 to the medical information management apparatus 4 and/or the medical information display apparatus 2 (hereinafter “sending state mark”) M6 is displayed in a “sending” section. The sending state mark M6 virtually expresses a sending state for the analysis result, namely sending completed, during sending, sending stopped, or sending failed. A user can know a sending state of the analysis result by looking at the sending state mark M6.

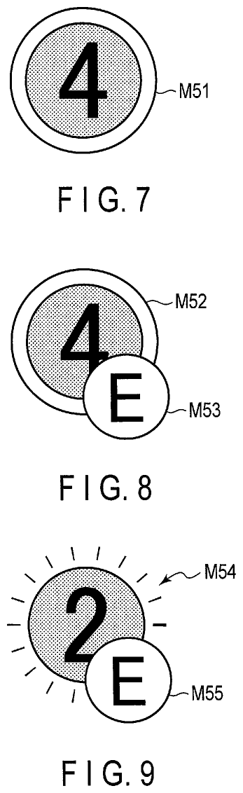

FIGS. 7, 8, and 9 are diagrams indicating examples of the summarized status mark. FIG. 7 is a diagram indicating a summarized status mark M51 relating to each of the third and fourth examinations on the list of FIG. 6. The summarized status mark M51 expresses that the total number of analysis processes belonging to the examination is four and all four analysis processes have been completed. Since no error mark (described later) is added to the summarized status mark M51, the mark expresses that all four analysis processes are successful.

FIG. 8 is a diagram showing the summarized status mark M52 relating to the first examination on the list of FIG. 6. The main mark M521 of the summarized status mark M52 shows that the total number of analysis processes belonging to the examination is four and all four analysis processes have been completed. To the main mark M521, an error mark M522 is added expressing that at least one analysis process of the four has failed. In other words, the summarized status mark M52 shows, as a whole, that the total number of analysis processes belonging to the examination is four, all four items are completed, and at least one of them failed.

FIG. 9 is a diagram showing a summarized status mark M53 relating to the second examination in FIG. 6. The main mark M531 of the summarized status mark M53 shows that the total number of analysis processes belonging to the examination is two, and at least one of two analysis processes is being performed. To the main mark M531, an error mark M532 is added expressing that at least one analysis process of the two has failed. In other words, the summarized status mark M53 shows, as a whole, that the total number of analysis processes belonging to the examination is two, and at least one analysis process is completed, and at least one of them failed.

As shown above, by displaying a summarized status on which performance states of the plurality of analysis processes included in one examination are summarized on the examination list display screen, it is possible to know the overall performance state of the plurality of analysis processes. The summarized status mark is designed to clearly express an overall performance state of a plurality of analysis processes included in one examination even in a relatively small space such as a display section for an examination record. By displaying a summarized status mark on each examination record, it is possible to know a performance state of overall analysis processing in each examination on the examination list display screen.

### Second Embodiment

As shown in FIG. 1, the medical information display apparatus 2 is accessible from a plurality of display terminals 5, and users having various purposes can view the management screen. As described above, results of all the analysis processes of an examination are displayed on the management screen; however, in reality, the roles and locations vary between users and an analysis process of interest varies between users. The medical information display apparatus 2 according to the second embodiment selects a display-targeted analysis process from all analysis processes in each examination in accordance with the role and/or location of the user, and displays a result of the display-targeted analysis process only. Hereinafter, the medical information processing system 1 according to the second embodiment will be described below. In the following description, structural elements having substantially the same functions as in the first embodiment will be denoted by the same reference symbols, and a repeat description will be given only where necessary.

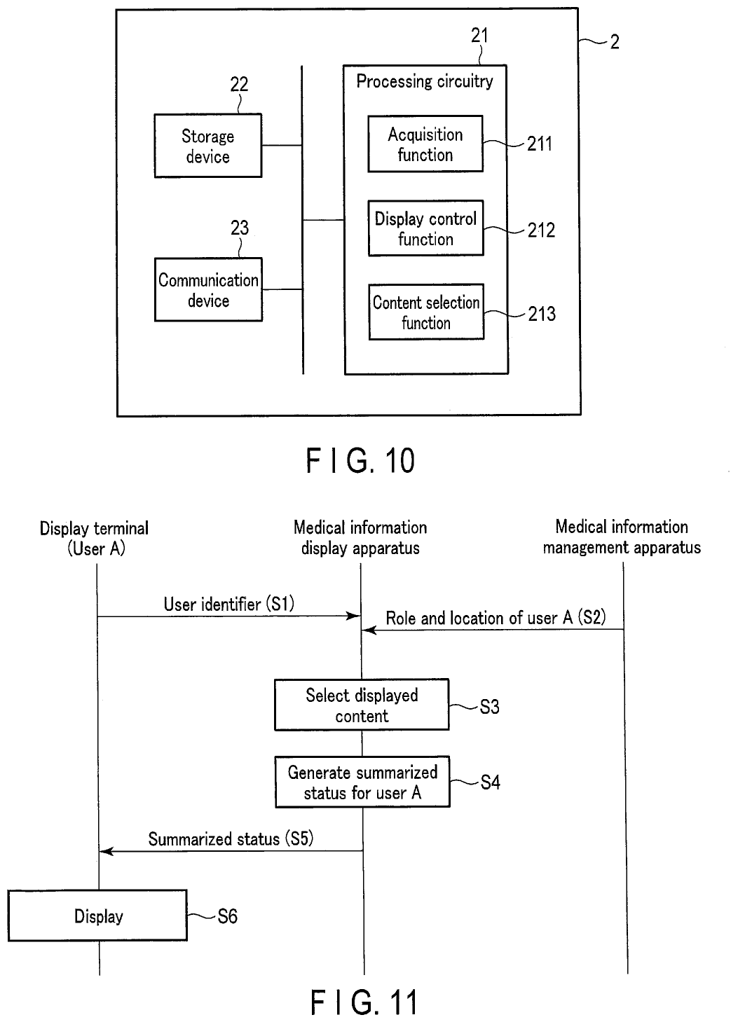

FIG. 10 is a diagram showing a configuration example of the medical information display apparatus 2 according to the second embodiment. As shown in FIG. 10, the processing circuitry 21 of the medical information display apparatus 2 according to the second embodiment realizes the content selection function 213 in addition to the obtainment function 211 and the display control function 212.

By realization of the content selection function 213, the processing circuitry 21 selects a display-targeted analysis process from all analysis processes in each examination in accordance with the role and/or location of the user. The role according to this example is a job that each user is in charge of in relation to analysis processing of a medical image in the medical information processing system 1. Examples of the role is an emergency doctor, radiologist, researcher and/or administrator, etc. The location according to this example means a residence area, a hospital, and/or an academic society, etc. that each user belongs to in relation to analysis processing of a medial image in the medical information processing system 1.

Through realization of the display control function 212, the processing circuitry 21 displays a result of the display-targeted analysis process. As an example, the processing circuitry 21 displays a summarized status indicating the number of analysis processes of a display target, and whether each processing item is being performed, has not yet been performed, is successful or has failed.

Hereinafter, an operation example of the medical information processing system 1 according to the first embodiment is described.

FIG. 11 is a diagram showing a flow of a summarized status display process by the medical information display system 1 according to the second embodiment. Assume user A is logged in using the display terminal 5.

As shown in FIG. 11, the display terminal 5 transmits a user identifier relating to user A to the medical information display apparatus 2 (step S1). The user identifier is transmitted at a timing at which the display terminal 5 transmits a display request for displaying the management screen to the medical information display apparatus 2. The medical information display apparatus 2 receives the user identifier relating to user A. The timing of transmitting a user identifier is not limited to the above-described example; a user identifier may be transmitted at a timing at which user A is logged into the medical information display apparatus 2 via the display terminal 5 or when the button for instructing transmitting a user identifier is pressed.

After step S1, the medical information display apparatus 2 transmits a sending request for data of the role and location of user A to the medical information management apparatus 4, and upon receipt of the sending request, the medical information management apparatus 4 transmits data of the role and location of user A to the medical information display apparatus 2 (step S2). Specifically, the medical information management apparatus 4 has a database in which user identifiers of a plurality of users are associated with their roles and locations (hereinafter, a “role and location database”). The medical information management apparatus 4 searches the role and location database using the received user identifier of user A as a search key to extract a role and a location associated with the user identifier of user A. The extracted data of the role and location of user A is transmitted to the medical information display apparatus 2. The medical information display apparatus 2 receives the data of the role and location of user A.

After step S2, the medical information display apparatus 2 selects display content of the management screen to be displayed on the display terminal 5 for user A (step S3). After step S3, the medical information display apparatus 2 generates a summarized status for user A (step S4). Specifically, in step S3, the processing circuitry 21 first selects display content based on the role and/or the location of user A. Specifically, the processing circuitry 21 selects one or more analysis processes of interest relating to the role and/or location of the user as a display-targeted analysis process from a plurality of analysis processes performed in a display-targeted examination. The method of selection is not particularly limited. As an example, the medical information management apparatus 4 has a database in which an identifier of an analysis process of interest is associated with each combination of a role and/or location (hereinafter, an “analysis processing database”). The identifiers of the analysis processes of interest may be determined by track record in the past or by inquiry, etc. The processing circuitry 21 searches the analysis processing database using a combination of the role and/or location of user A as a search key to extract an identifier of the analysis process associated with the combination. Then, in step S4, the processing circuitry 21 generates a summarized status of user A based on a result of the analysis process corresponding to the extracted identifier.

After step S4, the medical information display apparatus 2 transmits data of a summarized status generated in step S4 to the display terminal of user A (step S5). After step S5, the display terminal 5 receives data of user A's summarized status and displays the received summarized status on the examination list display screen, etc. (step S6).

Herein, the generation and display of a summarized status is specifically described.

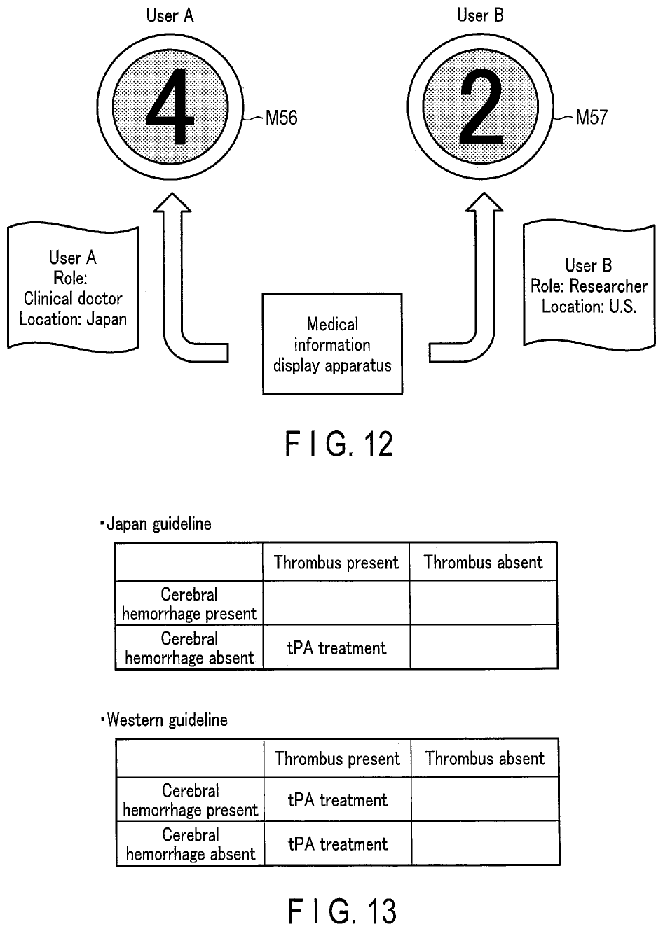

FIG. 12 schematically shows the process of setting display content for the summarized status. In FIG. 12, assume that a user A who is using the display terminal 5a and user B who is using the display terminal 5b use the medical information processing system 1. The role of user A is a doctor, and the area, which is a variation of the location, is Japan. The role of user A is a researcher and the location is the U.S. Furthermore, assume the total number of analysis processes performed in a display-targeted examination is five. As an example, if four of five analysis processes are the analysis processes associated with the role “doctor” and the location “Japan” of user A, the processing circuitry 21 generates a summarized status mark M56 of user A based on those four analysis processes. As another example, if two of five analysis processes are the analysis processes associated with the role “researcher” and the location “U.S.”, the processing circuitry 21 generates a summarized status mark M57 of user B based on those four analysis processes.

The method of generating the summarized status marks M56 and M57 is the same as that in the first embodiment. For example, if at least one of four analysis processes targeted for display in relation to user A has failed, an error mark may be added to the summarized status mark M56. The same applies to user B.

Herein, the display of a summarized status according to a role is described. An analysis process for which the user needs to wait for completion varies depending on a user's role. For example, an emergency doctor makes a determination after analysis processing using analysis apps that aid determination for acute treatment, such as “Hemorrhage” and “Ischemia”, is completed. Accordingly, for the role “emergency doctor”, an analysis process in which a determination for acute treatment is aided is set to a display target. A radiologist conducts diagnosis reading after a series of automatically performed analysis processes is completed. There is a case where an analysis app is run during diagnosis reading. Thus, for the role “radiologist”, all analysis processes are set to be a display target. A researcher checks the progress of a specific research-purpose analysis on the management screen. Or, a researcher waits for a notification of completion of the analysis. Thus, for the role “researcher”, analysis processes for researcher purposes are set to be a display target. An administrator checks on errors of all analysis apps and delays in running of the processing. Thus, for the role “administrator”, all analysis processes are set to be a display target.

Thus, since whether or not an analysis process is set to be a display target is determined based on a possibility of using an analysis process depending on a user's role, a summarized status displayed on the examination list can be dynamically changed in accordance with a user's role. Thus, for a user who has a role that does not require the user to wait for a completion of all analysis processes, such as an emergency doctor or a researcher, it is allowed to display a summarized status on which only the results of necessary analysis processes are summarized, and it is allowed to check analysis results immediately after the necessary analysis processes are completed.

If a user has multiple roles, a role targeted for generating a summarized status may be selected from the multiple roles of the user by a user via the display terminal 5.

Next, the display of a summarized status according to the location is described. Assume the location is an area. As an example where determination of the location is required, a case where a medicament that dissolves thrombus is injected as a treatment for acute cerebral infarction (hereinafter, a “tPA treatment”) is assumed. The administration criteria for determining whether or not a tPA treatment should be administered varies between areas, i.e., countries.

FIG. 13 is a diagram showing a presence/absence of performance of a tPA treatment under Japan guideline and western guideline. As shown in FIG. 13, whether or not a tPA treatment is administered is classified according to a presence/absence of a cerebral hemorrhage and a presence/absence of thrombus. In Japan, only when thrombus are present and a cerebral hemorrhage is absent can a tPA treatment be administered. In other words, only when both the analysis processing by the analysis app “Ischemia” for determining a presence/absence of thrombus and the analysis processing by the analysis app “Hemorrhage” for determining a presence/absence of a cerebral hemorrhage are completed is it possible to determine whether or not a tPA treatment should be administered. For this reason, for the location “Japan”, both of the analysis processing by the analysis app “Ischemia” and the analysis processing by the analysis app “Hemorrhage” are set to be display targets. In Europe and the U.S., on the other hand, if thrombus are present, a tPA treatment can be administered, regardless of a presence/absence of a hemorrhage. Thus, it is possible to determine whether or not a tPA treatment should be administered upon completion of the analysis processing by the analysis app “Ischemia”, and there is no need to wait for a completion of the analysis processing by the analysis app “Hemorrhage”. Thus, for the location “Europe and U.S.”, the analysis processing by the analysis app “Ischemia” is set as a display target.

Thus, since whether or not an analysis process is set to be a display target is determined based on a possibility of using an analysis process depending on a user's location, a summarized status displayed on the examination list can be dynamically changed in accordance with a user's location. Thus, for a user belonging to a location, such as Europe and the U.S., for which it is not necessary to wait for a completion of the analysis processing by the analysis apps “Ischemia” and “Hemorrhage”, it is necessary to summarize the results of only the analysis app “Ischemia”, and to check the analysis results immediately after the completion of the analysis app “Ischemia”.

If a user belongs to multiple locations, a location that is considered when a summarized status is generated may be selected from the multiple locations of the user by a user via the display terminal 5.

Next, the display of types of the analysis processing is described. The processing circuitry 21 selects, through realization of the content selection function 213, a display-targeted analysis process from a plurality of analysis processes, in accordance with a role and/or a location of a user who is using the display terminal 5. Through realization of the display control function 212, the processing circuitry 21 causes an icon expressing the display-targeted analysis process to be displayed. For example, the processing circuitry 21 causes an icon expressing the display-targeted analysis process to be displayed on the examination list display screen.

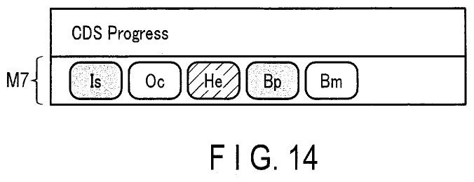

FIG. 14 is a diagram showing an example of displaying icons of analysis processing. As shown in FIG. 14, in the case of the examination “CDS Progress”, an icon M7 of a plurality of analysis processes performed in the examination “CDS Progress” is displayed. For example, of the icon M7, the icon “Is” is an icon expressing the analysis process by the analysis app “Ischemia”, and the icon “He” is an icon expressing the analysis process by the analysis app “Hemorrhage”.

The processing circuitry 21 causes the icon M7 expressing the display-targeted analysis process associated with the role and/or the location of the user in the role and location database to be displayed in the examination list display screen. Alternatively, the processing circuitry 21 may cause a window in which the icon M7 is placed and the examination list display screen to overlap each other or to be placed side by side. It is thus possible for the user to know the display-targeted analysis process of the user themselves.

As shown in FIG. 14, the processing circuitry 21 may display the icon M7 of the display-targeted analysis process to be displayed in a visually identifiable manner in accordance with a result of the processing. For example, an icon for an analysis process associated with a result “not yet performed”, such as the analysis apps “Is” and “Bp”, may be displayed in gray, an icon for a result “failed” such as the analysis app “He” may be displayed with diagonal lines, and an icon for a result “successful” such as the analysis apps “Oc” and “Bm” may be displayed normally. It is thus possible to know the result of each analysis process on the examination list display screen.

### First Modification

In the foregoing embodiment, the processing circuitry 21 changes the display content of a summarized status in accordance with the role and/or the location of the user. The processing circuitry 21 according to the first modification may change the display content of the analysis result display screen in accordance with a type of an analysis process. Hereinafter, the medical information processing system 1 according to the first modification is described. In the following description, structural elements having substantially the same functions as in the first and second embodiments will be denoted by the same reference symbols, and a repeat description will be given only where necessary.

As an example, the processing circuitry 21 according to the first modification causes, if an analysis process according to a manual performance is included in a plurality of analysis processes according to the display-targeted examination, a result of an analysis process according to a manual performance to be displayed only on the display terminal 5 of the user who instructed the manual performance. In other words, the processing circuitry 21 switches the display of a result of the manually-performed analysis process in accordance with whether or not the user is a user who instructed the manual performance.

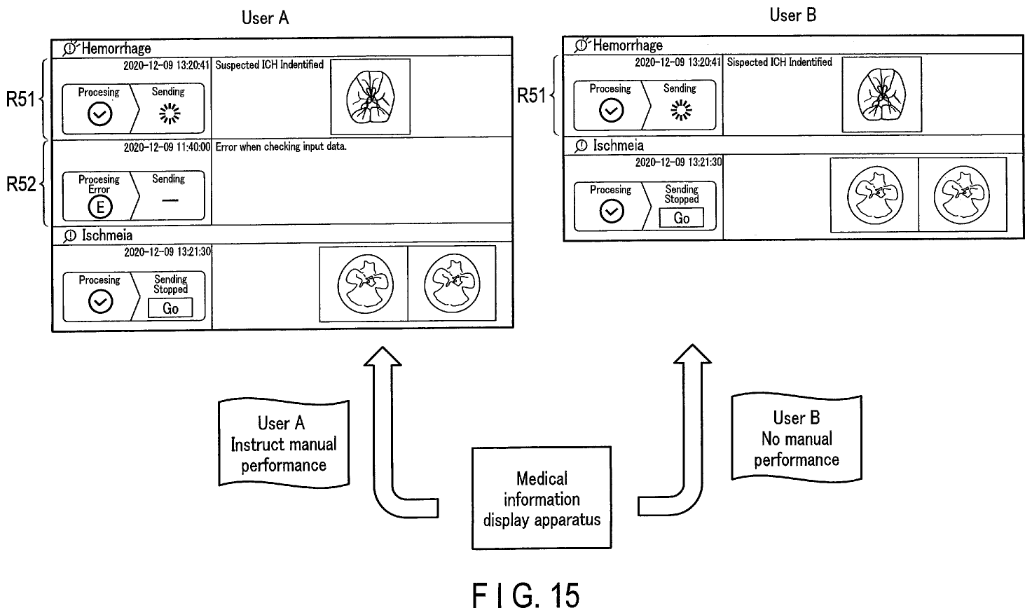

FIG. 15 is a diagram schematically showing changing of an examination result display screen between user A and user B. As shown in FIG. 15, the analysis process according to automatic running of the analysis app “Hemorrhage” is completed, and the display area R51 for a result of the analysis process is displayed. Herein, assume that user A instructed a manual performance of the analysis process by the analysis app “Hemorrhage”, and user B did not. In this case, the processing circuitry 21 causes the display terminal 5a of user A to display the display area R52 or a result of a manually performed analysis process by the analysis app “Hemorrhage” on the examination result display screen. The processing circuitry 21, on the other hand, does not cause the display terminal 5b of user B to display a display area R52 for a result of a manually performed analysis process by the analysis app “Hemorrhage” on the examination result display screen. It is thereby possible to display a result relating to a manual performance only to a user who instructed the manual performance and is interested in the result.

The processing circuitry 21 may cause the examination result display screen etc. to display a GUI (graphical user interface) component that instructs publication (hereinafter, a “publication button”). If the publication button is pressed, the processing circuitry 21 causes the examination result display screen of the user who pressed the publication button to display a display area of a result of a manually performed analysis process.

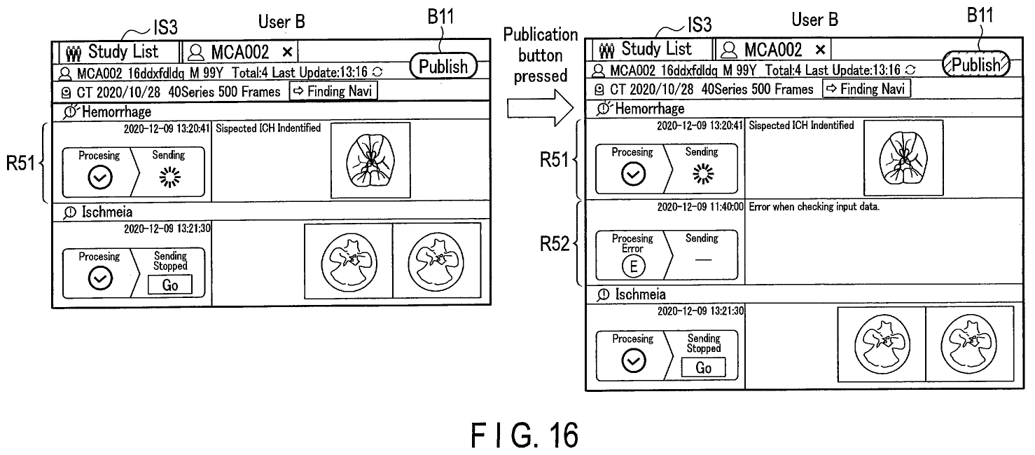

FIG. 16 is a diagram showing development of the examination result display screen IS3 regarding user B shown in FIG. 15 as a result of pressing a publication button. As shown in FIG. 16, a publication button B1 is displayed on the examination result display screen. If the publication button B1 is pressed by user B via the display terminal 5b, the processing circuitry 21 causes the examination result display screen of the display terminal 5b to display the display area R52 for a result of a manually performed analysis process. It is thereby possible to display a result of a manually performed analysis process to a user who wants to publish the result, separately from other results.

The display location of the publication button is not limited to a display terminal of a user who did not instruct a manual performance, such as user B. For example, the processing circuitry 21 may cause a display terminal of a user who instructed a manual performance, such as user A. User A may press the publication button when, for example, user A wants to publish a result of a manually performed analysis process to all users.

The processing circuitry 21 may determine whether or not a manually performed analysis process should be added to a processing target of a summarized status in accordance with whether the item is published or not. In other words, for a user for whom the manually performed analysis process is published, the processing circuitry 21 generates a summarized status in consideration of the manually performed item. On the other hand, for a user for whom the manually performed analysis process is not published, the processing circuitry 21 generates a summarized status, excluding the manually performed item.

The processing circuitry 21 according to the second embodiment causes the examination result display screen of each user to display only a result of an analysis processing time selected by the content selection function 213 according to the role and/or location of the user. The processing circuitry 21 according to the first modification may cause the publication button to be displayed on the examination result display screen of the user. It is preferable that the processing circuitry 21 cause results of all analysis processes relating to a display-targeted examination to be displayed when the publication button is pressed. For example, the processing circuitry 21 can select, through the content selection function 213, an analysis process compliant with the guideline adopted by the hospital or academic society to which user C belongs from a plurality of analysis process relating to a display-targeted examination, and by the display control function 212, the processing circuitry 21 can cause the display terminal of user C to display only a result of the selected analysis process. If user C wants a non-displayed result of an analysis process, the user needs to press the publication button. In this case, the processing circuitry 21 causes the display terminal of user C to display results of all analysis processes.

### Second Modification

The medical information display apparatus 2 according to the second modification presents to a user the running time and/or a remaining time of a plurality of analysis processes relating to a display-targeted examination. Hereinafter, the medical information processing system 1 according to this modification will be described below. In the following description, structural elements having substantially the same functions as in the first and second embodiments will be denoted by the same reference symbols, and a repeat description will be given only where necessary.

The processing circuitry 21 according to the second modification causes the display terminal 5 to display a running time and/or a remaining time of a plurality of analysis processes relating to the display-targeted examination. A running time means a predicted value of a time required from the start to the completion of the plurality of analysis processes. A remaining time means a length of time from a current point in time to the completion of the plurality of analysis processes. The remaining time is therefore equal to a value calculated by subtracting a process time up to a current point in time from the running time.

Herein, an example of a method of calculating a running time is described. The medical information management apparatus 4 stores a database in which a combination of a type of analysis process and processing conditions is associated with a separate running time. A separate running time means a running time of a single analysis process. As processing conditions, conditions that would affect a processing time, such as the number of frames, a data capacity, and an input parameter value, etc. of processing-targeted data, are recorded. A separate running time may be an actually recorded value or a predicted value statistically calculated based on an actually recorded value.

Before a performance of an analysis process relating to a display-targeted examination is started, the processing circuitry 21 obtains a type of analysis process and processing conditions for each of a plurality of analysis processes to be performed from the medical analysis apparatus 3, the medical information management apparatus 4, or the display terminal 5. The processing circuitry 21 searches the separate running time database using the obtained type of analysis process and processing conditions as search keywords for each analysis process, and extracts a separate running time associated with the search keywords. The processing circuitry 21 sums a plurality of separate running times respectively corresponding to a plurality of analysis processes to calculate a total running time of the plurality of separate running times.

A case where there are no processing conditions that match processing conditions designated as search keywords (hereinafter, “designated processing conditions”) in the running time database will be described. In this case, the processing circuitry 21 specifies a processing condition close to a designated processing condition from the running time database, and extracts a separate running time associated with the specified processing condition. The processing circuitry 21 may then linearly or non-linearly correct the extracted separate running time in accordance with a difference between a designated processing condition and a processing condition specified from the running time database.

When a running time is calculated, the processing circuitry 21 causes the display terminal 5 to display the calculated running time. A running time may be displayed in an examination record of a display-targeted examination in the examination list display screen. The display location of a running time is not limited to the above example, and a running time may be displayed at any location. The processing circuitry 21 then calculates a remaining time by subtracting a time elapsed since the commencement of the processing from the running time.

At this time, the processing circuitry 21 may dynamically change the running time for each user in accordance with the role and/or the location of the user when displaying the running time, similarly to the case of the summarized status. In other words, the processing circuitry 21, by realization of the content selection function 213, selects an analysis process as a display target according to the role and/or the location of the user who is using the display terminal 5 from a plurality of the analysis processes relating to a display-targeted examination, and through the display control function 212, the processing circuitry 21 causes the display terminal 5 that the user is using to display the running time and/or the remaining time of the display-targeted analysis process. At this time, the processing circuitry 21 may calculate a running time and/or a remaining time by comparing the combination of the type of displayed-targeted analysis processing target and processing condition with the separate running time database.

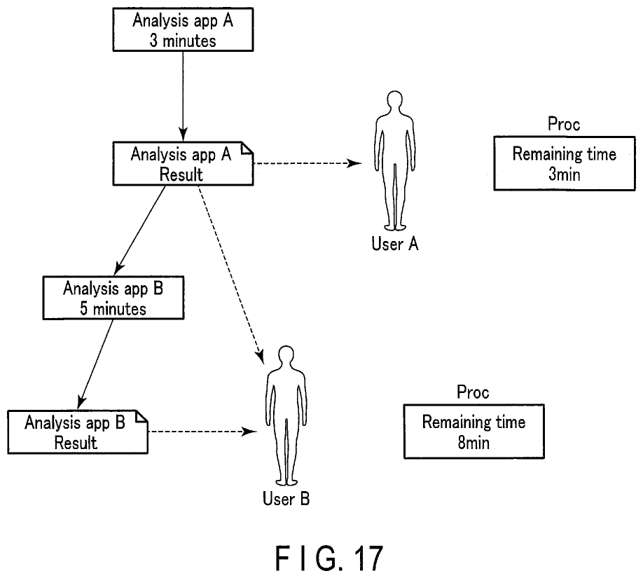

FIG. 17 is a diagram schematically showing an example of a display of a running time according to the second modification. As shown in FIG. 17, herein assume that an analysis process according to a role and/or a location of user A is an analysis process relating to the analysis app A, and an analysis process according to a role and/or a location of user B is an analysis process relating to the analysis app A and an analysis process relating to the analysis app B. Herein assume that the separate running time of an analysis process relating to the analysis app A is three minutes and the separate running time of an analysis process relating to the analysis app B is five minutes. In this case, before an analysis process is performed, the remaining time “three minutes” based only on the analysis app A is displayed in the Proc area (“processing area”) on the examination list display screen of user A's display terminal 5a. In the Proc area of the examination list display screen of user B's display terminal 5b, the remaining time “eight minutes” based on the analysis app A and the analysis app B is displayed. A real-time remaining time is displayed in accordance with an elapsed time since the commencement of the analysis processing. On the examination list display screen, a running time may be displayed instead of a remaining time, and both a remaining time and a running time may be displayed.

Thus, by displaying a total running time and/or a total remaining time of all analysis processes included in a display-targeted examination, a user is able to know a running time and/or a remaining time and to use time effectively. If display-targeted analysis processes are limited in accordance with a role and/or a location of a user, it is possible to know a running time and/or a remaining time based on an analysis process of interest of each user by displaying a running time and/or a remaining time based on the display-targeted analysis process.

### Others

According to the foregoing examples, the analysis processing of medical images is performed by the medical analysis apparatus 3. However, the present embodiment is not limited thereto. The analysis processing of medical images may be performed by the medical information display apparatus 2 or the medical information management apparatus 4. In the foregoing description, the medical images are managed by the medical information management apparatus 4; however, the management may be carried out by the medical information display apparatus 2 or the medical analysis apparatus 3. In the foregoing description, the medical information processing system 1 is a computer system having a medical information display apparatus 2, a medical analysis apparatus 3, a medical information management apparatus 4, and a display terminal 5; however, it may be a single computer that realizes the functions of the medical information display apparatus 2, the medical analysis apparatus 3, the medical information management apparatus 4, and the display terminal 5.

According to at least one of the foregoing embodiments, it is possible to improve viewability of analysis process results when the same analysis application is performed multiple times on medical images of the same examination.

The term “processor” used in the above description indicates, for example, a circuit, such as a CPU, a GPU, or an Application Specific Integrated Circuit (ASIC), and a programmable logic device (for example, a Simple Programmable Logic Device (SPLD), a Complex Programmable Logic Device (CPLD), and a Field Programmable Gate Array (FPGA)). The processor realizes its function by reading and executing the program stored in the storage circuitry. The program may be directly incorporated into the circuit of the processor instead of being stored in the storage circuit. In this case, the processor implements the function by reading and executing the program incorporated into the circuit. If the processor is for example an ASIC, on the other hand, the function is directly implemented in a circuit of the processor as a logic circuit, instead of storing a program in a storage circuit. Each processor of the present embodiment is not limited to a case where each processor is configured as a single circuit; a plurality of independent circuits may be combined into one processor to realize the function of the processor. Further, a plurality of components shown in FIG. 1, FIG. 2 and FIG. 10 may be integrated into one processor to achieve their functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information display apparatus comprising processing circuitry configured to:
obtain a plurality of execution results respectively corresponding to a plurality of analysis processes, the execution results being obtained by performing the plurality of analysis processes relating to at least one type of analysis application on a plurality of series of medical images; and
display a first display area for displaying the plurality of execution results which are summarized by a type of analysis application, and a second display area for displaying input images provided to the plurality of analysis processes on a display, the input images being identifiably displayed for each type of analysis application, the second display area differing from the first display area.

2. The medical information display apparatus of claim 1, wherein
the processing circuitry causes the first display area to display, as the plurality of execution results, identification information indicating that the analysis process is being performed, has not yet been performed, is successful, or has failed.

3. The medical information display apparatus of claim 1, wherein
the processing circuitry causes the first display area to display an analyzed image, which is an execution result of performing analysis processing, and/or input parameters of analysis processing, as the plurality of execution results.

4. The medical information display apparatus of claim 1, wherein
the processing circuitry causes the second display area to display a plurality of representative images respectively corresponding to the plurality of series, with an emphasis on a representative image of a series on which an analysis process selected from the plurality of analysis processes is performed.

5. The medical information display apparatus of claim 1, wherein
the processing circuitry causes the first display area and the second display area to be displayed on one screen, side by side.

6. The medical information display apparatus of claim 1, wherein
the processing circuitry is configured to:
display a third display area in which a list of examination records respectively corresponding to a plurality of examinations is displayed; and
display the first display area and the second display area relating to the single examination record if a single examination record is selected from the plurality of examination records.

7. The medical information display apparatus of claim 6, wherein
each of the plurality of examination results includes a performance state of each of the analysis processes, and
for each of the examination records, the processing circuitry display a mark summarizing the plurality of performance states relating to the examinations.

8. The medical information display apparatus of claim 7, wherein
the mark indicates the number of the analysis processes and whether each analysis process is being performed, has not yet been performed, is successful, or has failed.

9. The medical information display apparatus of claim 7, wherein
the processing circuitry is configured to:
select a display-targeted analysis process from the plurality of analysis processes in accordance with a role and/or a location of a user who is using the display terminal; and
display the mark indicating the number of display-targeted analysis processes and whether the display-targeted analysis process is being performed, has not yet been performed, is successful, or has failed.

10. The medical information display apparatus of claim 9, wherein
the role includes an emergency doctor, a radiologist, a researcher, and/or an administrator.

11. The medical information display apparatus of claim 9, wherein
the location includes a residential area, a hospital that the user works for, and/or an academic society.

12. The medical information display apparatus of claim 1, wherein
the processing circuitry is configured to:
select a display-targeted analysis process from the plurality of analysis processes in accordance with a role and/or a location of a user who is using the display terminal; and
display an icon expressing the display-targeted analysis process.

13. The medical information display apparatus of claim 1, wherein
if a manually performed analysis process is included in the plurality of analysis processes, the processing circuitry display a first result of the manually performed analysis process of the plurality of results only on the display of a first user who instructed the manual performance.

14. The medical information display apparatus of claim 13, wherein
the processing circuitry is configured to:
display a GUI component with an instruction to allow the first result viewable on the display of a second user differing from the first user to; and
display the first result on the display of the second user when the GUI component is pressed.

15. The medical information display apparatus of claim 1 wherein
the processing circuitry is configured to:
select an analysis process suitable for a location of a user who is using the display from the plurality of analysis processes; and
display a result of the selected analysis process on the display of the user.

16. The medical information display apparatus of claim 1, wherein
the processing circuitry is configured to:
select an analysis process that is compliant with guideline adopted by a hospital or an academic society to which a user using the display terminal belongs.

17. The medical information display apparatus of claim 1, wherein
the processing circuitry displays a running time and/or a remaining time of the plurality of analysis processes.

18. The medical information display apparatus of claim 6, wherein
for each of the plurality of examination records, the processing circuitry displays a mark indicating a sending state of an analysis result of each of the plurality of analysis processes relating to an examination corresponding to the examination record.

19. The medical information display apparatus of claim 17, wherein
the processing circuitry is configured to:
select a display-targeted analysis process from the plurality of analysis processes in accordance with a role and/or a location of a user who is using the display; and
display a running time and/or a remaining time of the display-targeted analysis process among the plurality of analysis processes on the display that is being used by the user.

20. A medical information display method comprising:
obtaining a plurality of execution results respectively corresponding to a plurality of analysis processes, the execution results being obtained by performing the plurality of analysis processes relating to at least one type of analysis application on a plurality of series of medical images; and
display a first display area for displaying the plurality of execution results which are summarized by a type of analysis application, and a second display area for displaying input images provided to the plurality of analysis processes on a display, the input images being identifiably displayed for each type of analysis application, the second display area differing from the first display area.
